(19) United States
(12) Patent Application Publication
(10) Pub. No.: US 2023/0201615 A1
(43) Pub. Date: Jun. 29, 2023

(54) NON-INTRUSIVE DELIVERY MECHANISM FOR PRODUCING PHYSIOLOGICAL EFFECTS IN LIVING ORGANISMS

(71) Applicant: EMulate Therapeutics, Inc., Bellevue, WA (US)

(72) Inventors: Xavier A. Figueroa, Seattle, WA (US); Bennett Michael Butters, Lacey, WA (US)

(21) Appl. No.: 18/088,354

(22) Filed: Dec. 23, 2022

Related U.S. Application Data
(60) Provisional application No. 63/294,054, filed on Dec. 27, 2021.

(51) Int. Cl. A61N 1/40; A61B 5/00; A61B 5/01

(52) U.S. Cl. CPC A61N 1/40; A61B 5/4848; A61B 5/01

(57) ABSTRACT

Systems and methods for producing physiological effects in response to simulated stimuli on living organisms are disclosed herein. In one example, a non-intrusive delivery of drug-simulating signals causes physiological effects on a living organism.

### CROSS-REFERENCE TO RELATED APPLICATION(S)

The present application claims the benefit of, and priority to, U.S. Provisional Patent Application No. 63/294,054 filed Dec. 27, 2021, which is herein incorporated by reference in its entirety.

### BACKGROUND

Living organisms continuously experience various physiological effects under typical and atypical conditions. For example, a person can respond to stress induced from external stimuli by producing certain hormones that affect the way the person's body functions. The person can shift from one physiological state to another as the person's environment changes or as stimuli change. The stimuli can have a beneficial physiological effect. For example, a person can consume medication or experience physical interactions that mitigate the negative symptoms of various diseases and adverse health conditions. For example, Diazepam is a drug that, when consumed, can have an effect on a person's physiology to reduce anxiety, seizures, etc. The medication can be taken by mouth, inserted into the rectum, injected into muscle, injected into a vein, or used as a nasal spray. The effects of the drug can begin at different times depending on the form in which the medication is consumed.

One reason why there are different ways to consume medicines is because the effects of a drug can depend on the way in which the drug is processed by a particular person. For example, when administered into a vein, the effects of a drug can begin within one to five minutes and last for an hour. By mouth, effects can begin between 15 to 60 minutes after the drug is administered. Moreover, some people may prefer one form of consumption or have a physical aversion to a form. Still, there remains a desire to avoid consuming the medication altogether and still experience the same physiological benefits. Moreover, any medication has side effects such as, suicidal tendencies, decreased breathing, agitation, etc. Accordingly, it would be desirable to experience all the benefits of medication while reducing or preventing its side effects.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present technology. Accordingly, various elements may be arbitrarily enlarged to improve legibility. For ease of reference, throughout this disclosure identical reference numbers may be used to identify identical, or at least generally similar or analogous components or features.

FIG. 1 illustrates a system including wearables for delivering drug-simulating signals to a person or target to simulate a desired physiological effect;

FIGS. 2A and 2B illustrate diagrams of a cable and coil assembly during manufacture;

FIG. 3 illustrates an implementation of a connector core of a cable and coil assembly;

FIG. 4 illustrates a method of manufacturing a coil and cable assembly;

FIG. 5 illustrates a method for enhancing a drug-simulating signal;

FIG. 6 illustrates a method of operating a system configured to provide magnetic field therapy that is non-invasive, non-thermal, and mobile;

FIG. 7 illustrates a Helmholtz configuration including coils that can target drug-simulating signals toward an object between the coils;

FIG. 8 is a diagram that illustrates a non-uniform array of devices including signal generators located in a vehicle;

FIG. 9 is a block diagram that illustrates a system including machine learning (ML) capabilities that improve the efficacy of drug-simulating signals; and

FIG. 10 is a block diagram that illustrates an example of a computer system in which at least some operations described herein can be implemented.

### DETAILED DESCRIPTION

The disclosed technology includes a non-intrusive delivery mechanism for producing a physiological effect on a living organism. The technology overcomes the drawbacks of prior methods for delivering a medicine or other substance which has a physiological effect when ingested or otherwise introduced into the target organism (“body”). The drawbacks of prior methods include being intrusive and the substances themselves can cause undesirable side-effects. The disclosed technology includes electromagnetic signals that are radiated on a tissue of an organism to simulate the physiological effects of the recorded compound on the body. The recorded signals are referred to herein as “drug-simulating signals” or “cognates,” which are designed to simulate the electrostatic potential of certain molecules or compounds in the body that are produced in response to the physical molecules that were recorded. That is, a cognate includes a signal that approximately reproduces magnetic fields that emanate from one or more predetermined chemical, biochemical, and/or biological molecules.

More generally, a system can record signals associated with chemical, biochemical, or biological molecules or from chemical, biochemical, or biological agents. The recordings represent the electrostatic potential of the chemical, biochemical, or biological molecules or agents used to provide a physiological effect. In one implementation, a magnetometer can detect magnetic fields. The movement of electrical charge produced by a molecule produces a magnetic field that is detectable by the magnetometer. The magnetic field induces a voltage within a superconducting quantum interference device (SQUID) amplifier and maintains a voltage from that point on, which can be recorded.

In some cases, a substance can counteract an ailment or other adverse health conditions. In one implementation, transduction technology is coupled through Bluetooth (or other communication technology) to a device including biometric sensor technology such as a smart watch. In one implementation, a stand-alone biosensor could be monitoring a living organism's sleep pattern and through a Bluetooth connection it can select an appropriate signal or cognate and play it to achieve a desired effect, such as better sleep. In another implementation, a biosensor detected a biometric signature for anxiety, exemplified by an increase in heart rate, pulse, and blood pressure. The sensor could communicate with the transduction device to deliver the appropriate signal or cognate to reduce anxiety.

The systems and methods disclosed herein may be configured to produce, or emulate, a physiological effect similar to that caused by the delivery of a chemical, biochemical, or biologic agent to a person or other target but without the use of drugs. For example, techniques can involve generating an efficacious field including electromagnetic or magnetic fields, that simulate signals of chemicals, biochemical, or biologics. Thus, the systems and methods of the present technology will allow a person to receive an electronic “prescription” or dosage of electromagnetic or radio frequency (RF) energy with, for example, the click of a button. Therefore, the described technology is non-invasive, non-thermal, and can be mobile.

As used herein, the term “drug” can broadly refer to any chemical, biochemical or biologic molecules including medicines, proteins, RNA and DNA sequences, or any other substance or agent that can cause a physiological effect. Further, as used herein, and described in more detail below, the terms “magnetic field,” “electromagnetic field,” and the like are used interchangeably to refer to targeting energy to a selected region of a living being so as to produce a physiological effect that can, for example, provide beneficial effects or address adverse health effects. Moreover, the RF energy has a characteristic that simulates the effect of a specific drug.

In some implementations, the induced physiological effects can mitigate pain or alleviate disorders of the central nervous system (CNS) such as depression, anxiety, post-traumatic stress disorder, and even alleviate symptoms of movement disorders. The drug-simulating signals can produce other physiological effects such as that of caffeine or other stimulants or depressants. Moreover, a combination of drug-simulating signals can be combined to provide a desired physiological effect.

The intensity of the signal can be adjusted up or down within a range that provides an efficacious field. That is, only a narrow range for a signal can produce an efficacious effect such that increasing the intensity above an upper threshold or decreasing the intensity below a lower threshold will not cause a body to produce the desired physiological effect. This can be true regardless of body shape and size. For example, the same narrow range of intensity is required to produce a desired physiological effect for both a man that weighs 200 pounds or a woman that weighs 150 pounds.

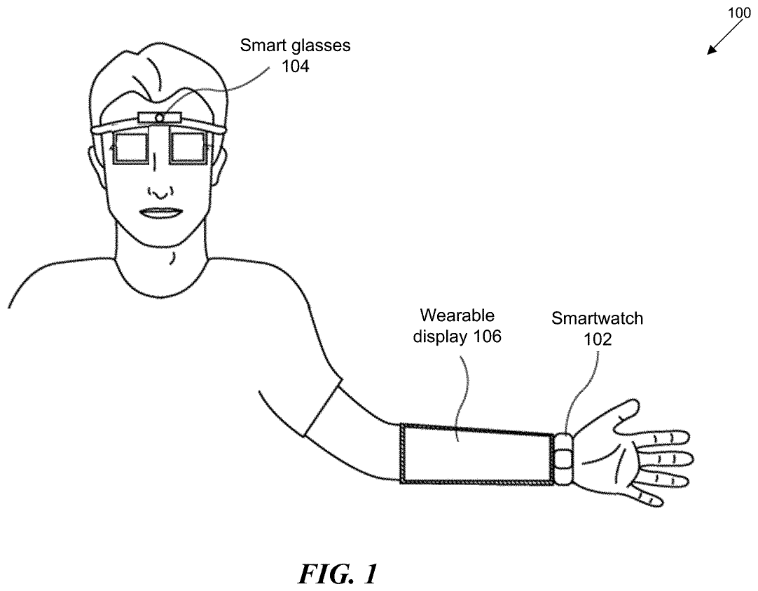

FIG. 1 illustrates a system 100 including wearable devices (hereinafter “wearables”) for delivering drug-simulating signals to a person to cause a desired physiological effect. As shown, the wearables can operate as a system, or individually, to selectively induce the effects of a drug such as an antidepressant. As such, the wearables can induce the physiological effects of that psychotropic drug by applying electromagnetic or magnetic fields to one or more areas of the living body. The fields are induced or generated to expose a particular area of the body with signals that simulate the drug. Of course, while a human is shown, the technology discussed, herein, can be used with other organisms such as animals to provide a pleasurable experience, alleviate ailments, etc. The observation of biological signals that are used to simulate drugs is discussed in great detail in patent applications and patents that are co-owned by the assignee of the instant application. These patents and applications include U.S. Pat. Nos. 6,724,188; 6,995,558; 6,952,652; 7,081,747; 7,412,340; and 7,575,934; 9,417,257; 10,046,172; 11,103,721; and PCT Application No. PCT/US2009/002184, each of which is hereby incorporated by reference in entirety.

The wearables can provide various advantages over intrusive forms of delivering substances that cause desired physiological effects. For example, wearables are portable and allow the person to receive therapy while at home, at work, at school, and during recreation. Furthermore, the wearables can enable a person to simulate the effect of receiving the drug without visiting a health care facility, without incurring extensive recovery time, and possibly without experiencing side-effects such as, for example, nausea, fatigue, loss of appetite, and the development of infections. Moreover, the signals can be tuned to optimize the physiological effects in a manner that is not readily possible with conventional drugs.

As shown, the wearables can be component parts that operate collectively or independently to produce a physiological effect. The component parts include different wearables, which are smart electronic devices (e.g., include micro-controllers) that are worn close to and/or on the surface of the skin, where they detect, analyze, and transmit information concerning, for example, body signals (e.g., vital signs) and/or ambient data and which can allow biofeedback to the wearer. Wearables such as activity trackers are an example of Internet of Things (IoT) devices that include electronics, software, sensors, and/or connectivity that enable objects to exchange data over the internet with a manufacturer, operator, and/or other connected devices, without requiring human intervention.

Wearables can be used to collect data of a user's health such as heart rate, calories burned, steps walked, blood pressure, release of certain biochemicals, time spent exercising, seizures, physical strain, etc. The data collected by a wearable can be used to, for example, forecast changes in mood, stress, and health; measure blood alcohol content; measure athletic performance; monitor how sick the user is; conduct long-term monitoring of a person with heart and circulatory problems; and perform health risk assessments. This data can be processed to select one or more cognates, and to adjust the amount of power applied and the duration of exposure of drug-simulating signals delivered to the person. Moreover, the wearables can deliver the signals instantaneously and dynamically change the “dosage” radiated on the body (e.g., adjust duration of exposure to drug-simulating signals).

In the illustrated example, the wearables include a smartwatch 102, smart glasses 104, and a wearable display device 106 on the forearm. The smartwatch 102 can provide a local touchscreen interface for daily use, while an associated mobile app on a smartphone (not shown) provides for management and telemetry (e.g., biomonitoring). The smartwatch 102 can include apps, a mobile operating system, and WiFi/Bluetooth connectivity. Smart glasses 104 add information alongside of what the wearer sees. Alternatively, smart glasses 104 can change their optical properties at runtime. Superimposing information onto a field of view is achieved through an optical head-mounted display (HMD) or embedded wireless glasses with transparent heads-up display (HUD) or augmented reality (AR) overlay. Modern smart glasses are effectively wearable computers which can run self-contained mobile apps. Some are handsfree and can communicate with the internet via natural language voice commands, while others use touch buttons.

In one example, the smart glasses 104 are part of a headgear that can be used to position a signal generator (e.g., coil) around the cranium of a person. The headgear can include a breathable mesh, elastic straps, and a band that can provide a comfortable apparatus for carrying, securing, or otherwise positioning the coil around the cranium of the person. The headgear may also include fasteners for securing the band over the coil. The fasteners may be influenced with Velcro, snaps, or other types of securing devices.

The wearable display device 106 can function like a smartphone to combine a mobile telephone and computing functions into one unit. It is distinguished from other smart devices by its stronger hardware capabilities and extensive mobile operating systems, which facilitate wider software, internet, and multimedia functionality, alongside core functions. The wearables can contain a number of integrated circuit (IC) chips, including various sensors such as a magnetometer, proximity sensors, barometer, gyroscope, accelerometer and more, and support wireless communications protocols (e.g., Bluetooth, Wi-Fi, or satellite navigation).

In accordance with implementations, a wearable or other device can be secured to the person by using fasteners, such as tape, elastic bandages, gauze, or the like. A system including wearables can include a controller and a battery charging device. For various security reasons, each component may be manufactured so that a housing cannot be opened easily. To allow a person to continuously experience a cognate's effect, one or more additional components are provided to allow the person to receive the effect while any of the wearables are inoperable. In one example, the components of the system are disposable, or the system as a whole with the one or more components is disposable.

In one implementation, each component contains a signal generator for generating an efficacious field including electromagnetic signals that are directed to the location of the person where the particular component is worn. In one example, a signal generator includes a coil and/or transmitter with one or more conductors configured to generate a magnetic or electromagnetic field to produce drug-simulating signals that simulate the physiological effect of the drug on a living person. The signal generator may be configured to have various electromagnetic characteristics. Additionally, the signal generator may be enclosed in a plastic or other composite material to both protect the windings of the coil (e.g., signal generator) and to provide a comfortable interface for the wearer. The signal generator can be flexible and malleable, can have a variety of shapes, can have different sizes or types, and can also include rigid coils. Advantageously, these signal generators can be externally secured to a person to provide drug-simulating signals, as opposed to subcutaneous insertion into a person.

The signal generator can have different shapes and/or sizes. For example, signal generators can include a small circular encapsulated coil, a large circular encapsulated coil, a rectangular encapsulated coil, and/or a substantially square encapsulated coil. Each shape may provide advantages for affecting particular parts of the body of the person. A variety of dimensions for the signal generator can be manufactured to more effectively apply drug-simulating signals to areas that vary in size. Each of the signal generators can have inner and/or outer diameters or lengths, ranging from just a few centimeters to several feet, according to various implementations.

In one implementation, a wearable has a cable that connects a coil to a controller to transmit various signals to the coil. The cable can include two or more conductors and a strength-providing member. Each of the conductors and members can perform a particular function. For example, some conductors can be electrically coupled to either end of the coil to enable current to flow to and from the coil to activate, stimulate, induce, or otherwise excite the coil. A shield conductor can be coupled to a ground and be configured to provide electromagnetic shielding for conductors. The strength member can be anchored to the coil and to a connector to provide strain relief to the conductors. In some implementations, the strength member is manufactured with a shorter length than the other conductors so that the strength member receives a majority of any strain applied between the coil and the connector.

The system and/or its component parts can communicate using encryption to, for example, thwart hacking. That is, the system can implement several types of encryption protocols to protect signal data. In one example, the system uses asymmetric encryption employing key pairs, a private key and public key. Symmetric encryption could also be used employing the same key for encryption and decryption, but could potentially be less secure. Further, hashing can be used to confirm the integrity of the signal data. Hashing generates a fixed length value associated with a file including a recording of a signal.

A file for a wearable to process to generate drug-simulating signals or the signals themselves can include metadata to aid in pre-processing or post-processing. For example, a file or signal can include an indication of the source of the signal, an expected physiological effect, or other variables such as information regarding sample preparation, concentration, and/or solvent data in the header information for each recording. Other metadata that can be included in the header includes tracking serial numbers and variables tweaked to optimize signals.

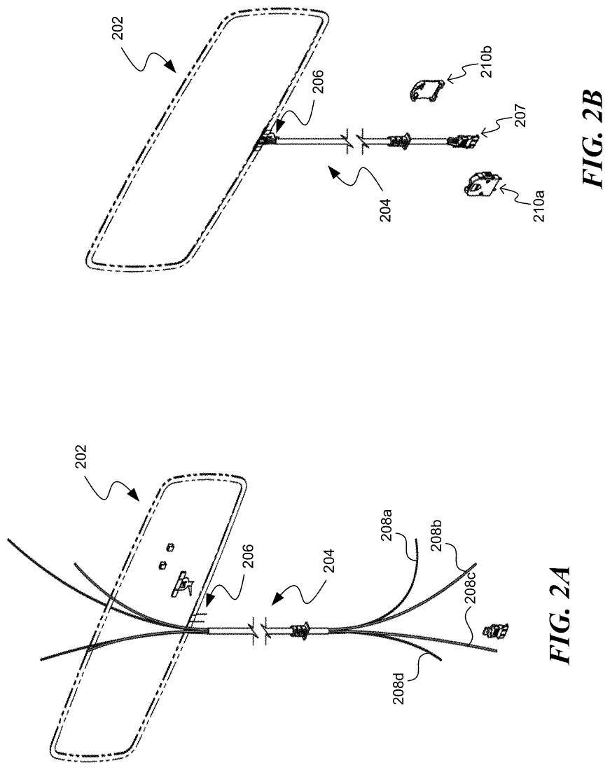

A wearable can embed a signal generator (e.g., coil) that is coupled to a controller via a cable. All or some of these components can be included in or external to the wearable. For example, FIGS. 2A and 2B illustrate diagrams of a cable and coil assembly during manufacture. The cable 204 connects a coil 202 to a connector 206 that enables a controller (not shown) to transmit various signals to the coil 202 through the cable 204. The cable 204 includes conductors 208a, 208b, a shield 208c, and a strength-providing member 208d (collectively “conductors 208”). Each of the conductors 208 are configured to perform a particular function. For example, conductors 208 can be electrically coupled to either end of the coil 202 to enable current to flow to and from the coil 202 to activate, stimulate, induce, or otherwise excite the coil 202. Shield conductor 208c can be coupled to ground and be configured to provide electromagnetic shielding for the conductors 208a and 208b. Strength member 208d can be anchored to the coil 202 and to the connector 206 to provide strain relief to the conductors 208a-208c. In some implementations, the strength member 208d is manufactured with a shorter length than the other conductors so that the strength member 208d receives a majority of any strain applied between the coil 202 and the connector 206.

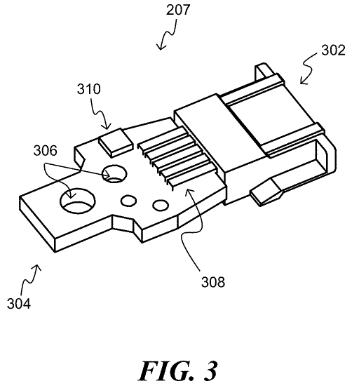

As illustrated in FIG. 2B, the connector 206 has three parts, (1) the connector core 207, (2) a connector housing 210a, and (3) a second connector housing 210b. The connector housings 210a and 210b encapsulate the connector core 206 to protect traces and electronic devices carried by the connector core 207. FIG. 3 illustrates an implementation of the connector core 207. The connector core 206 has a controller end 302 and a cable end 304. The controller end 302 is configured to mateably couple to the controller, and the cable end 304 is configured to provide an interface for the conductors 208. In some implementations, the strength member 208d is anchored to one or more holes 306 to provide strain relief. The conductor core 207 may also carry traces 308 to which the conductors 208 are electrically coupled to facilitate communication with the controller.

As a security feature of the coil and cable assembly, the connector core 207 can carry an integrated circuit 310. The integrated circuit 310 can be a microprocessor or may be a stand-alone memory device. The integrated circuit 310 can be configured to communicate with the controller through the controller end 302 using communication protocols such as I2C, 1-Wire, and the like. The integrated circuit 310 may include a digital identification of the coil with which the connector core 306 is associated. The digital identification stored on the integrated circuit 310 may identify electrical characteristics of the coil, such as impedance, inductance, capacitance, and the like. The integrated circuit 310 may also be configured to store and provide additional information such as the length of the conductor of the coil, physical dimensions of the coil, and number of turns of the coil.

In some implementations, the integrated circuit 310 includes information to prevent theft or reuse in a knock-off system, such as a unique identifier, cryptographic data, encrypted information, etc. For example, the information on the integrated circuit 310 may include a cryptographic identifier that represents measurable characteristics of the coil and/or the identification of the integrated circuit. If the cryptographic identifier is merely copied and saved onto another integrated circuit, for example, by an unauthorized manufacturer of the coil and cable assembly, the controller may recognize that the cryptographic identifier is illegitimate and may inhibit signal transmissions. In some implementations, the integrated circuit stores one or more encryption keys, digital signatures, stenographic data or other information to enable communications and/or security features associated with public key infrastructure, digital copy protection schemes, etc.

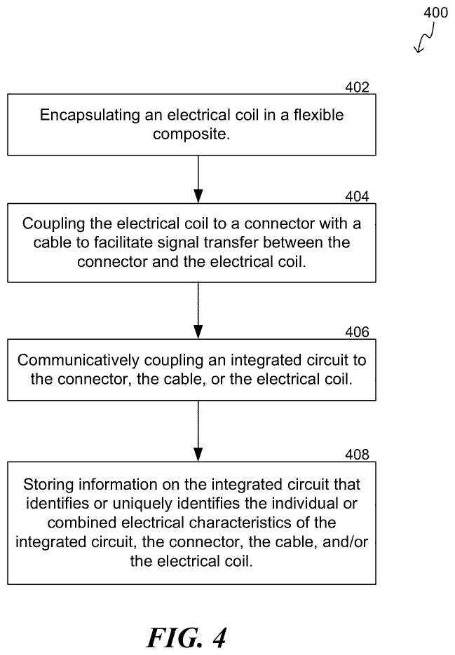

FIG. 4 illustrates a method 400 of manufacturing a coil and cable assembly, e.g., the coil and cable assembly, for use in providing a system that is non-invasive, non-thermal, and mobile.

At block 402, an electrical coil is encapsulated in a flexible composite. The flexible composite allows the electrical coil to be comfortably secured to the body of the patient to provide magnetic field therapy (e.g., drug-simulating signals).

At block 404, the electrical coil is coupled to a connector through a cable to facilitate secure transfer between the connector and the electrical coil. The cable may include multiple conductors that deliver signals between the connector and the electrical coil while providing mechanical strain relief to the signal carrying conductors.

At block 406, an integrated circuit is coupled to the connector, the cable, or the electrical coil. The integrated circuit may be coupled, for example, to the connector via one or more electrical conductors that may or may not also be coupled to the electrical coil. In block 406, other components could be added, integrated or coupled to the coil, connector and integrated circuit, such as smart eyewear components.

At block 408, information is stored to the integrated circuit that identifies or uniquely identifies the individual or combined electrical characteristics of the integrated circuit, the connector, the cable, and/or the electrical coil. The information may be a hash or other cryptographically unique identifier that is based on information that can be unique to the integrated circuit and/or the remainder of the coil and cable assembly. This security feature can be used to prevent or deter unauthorized remanufacture of coil and cable assemblies that are compatible with the controller for the therapy system. Additional security features are described herein (e.g., in connection with the operation of the controller for the therapy system).

The coil and cable assembly can be encapsulated in wearables so that the wearables operate as a closed system to deliver drug-simulating signals to a living organism. The delivery of the drug-simulating signal by the closed system is controlled by a program stored in a memory of the wearables. Further, the program is operable to respond to feedback collected by the wearables. In certain implementations, the closed system includes an amplifier circuit. The closed system allows at least a portion of the amplifier circuit's output signal to enter back into its input, creating feedback. Such an implementation can improve control of the output. For example, when the actual output of the amplifier circuit is compared to a desired output, the comparison may be used to finetune the output to achieve the desired output.

In other implementations, the amplifier circuit is programmable to adjust an intensity and/or an amplitude of the drug-simulating signal transmitted to the living organism. In such an implementation, the signal can be maintained at a certain range or adjusted to achieve a desired physiological effect in the living organism. Moreover, feedback will allow the closed system to dynamically adapt to any necessary requirements for achieving a desired physiological effect.

Alternatively, a handheld device (e.g., smartphone) can couple to the wearables wirelessly and control the signal generator in the wearables to deliver drug-simulating signals. Thus, the signal generator assembly embedded in the wearables can couple wirelessly to other devices as part of a system for producing physiological effects based on drug-simulating signals.

### System Controller

The controller can provide an interface to a person, to distribute and regulate drug-simulating signals to the signal generator, and to prevent unauthorized copying and/or distribution of the drug-simulating signals. According to various implementations, the controller can include various features such as a housing, a processor, memory, visual and audio interfaces, in addition to other features that are not illustrated or described herein for the sake of brevity. In one implementation, a microcontroller circuitry is configured for operating the controller. The circuitry can include a microprocessor, a reset circuit, and a volatile memory. The microcontroller can be a standard microprocessor, microcontroller or other similar processor, or alternatively be a tamper-resistant processor to improve security. The microprocessor can include a number of analog and/or digital communication pins to support communications with electronics that are both external and internal to the housing. The microprocessor may include: (1) a USB connector to support communication via the USB protocol, (2) display connectors to communicate with a visual interface, and (3) audio connectors to provide an audio interface, in addition to other data communication pins.

The microcontroller can securely receive prescription files from one or more external devices via the connectors or wirelessly. Encryption of the prescription file increase security of the contents of prescription file. Encryption systems regularly suffer from what is known as the key-distribution-problem. The standard assumption in the cryptographic community is that an attacker will know (or can readily discover) the algorithm for encryption and decryption. The key is all that is needed to decrypt the encrypted file and expose its private content. The legitimate user of the information must have the key. Distribution of the key in a secure way attenuates the key-distribution-problem.

In some embodiments, the microcontroller is configured to use the Advanced Encryption Standard (AES). AES is a specification for the encryption of electronic data established by the U.S. National Institute of Standards and Technology (NIST) and is used for inter-institutional financial transactions. It is a symmetric encryption standard (the same key is used for encryption and decryption) and can be secure while the key distribution security is maintained. In some implementations, the microcontroller uses a 128-bit AES key that is unique to each controller and is stored in non-volatile memory. The encryption key can be random to reduce the likelihood of forgery, hacking, or reverse engineering. The encryption key can be loaded into non-volatile memory during the manufacturing process or before the controller is delivered to customers (physicians or patients). Using AES encryption, the prescription file can be encrypted and uploaded to one or more servers to facilitate selective delivery to various controllers.

For example, a physician or other medical professional can obtain authorization to download prescription files to controllers for his/her patients. When the physician contacts and logs in to a server to obtain a prescription file, the physician may first need to provide certain information, e.g., may need to identify the target device (the controller), for the server (e.g., by a globally unique ID (GUID) stored in the controller) so that the server can look up the target device in a database and provide a prescription file that is encrypted with a key that is compatible with the controller. The encrypted prescription file can then be loaded into the non-volatile memory via the microcontroller, using USB or another communications protocol. Alternatively, or additionally, the encrypted prescription file may be stored directly to the non-volatile memory during the manufacturing process to reduce the likelihood of interception of the prescription file, and before the front and back portions of the housing are sealed together.

The microcontroller can also be configured to log use of the therapy system by a patient. The log can be stored in a non-volatile memory and downloaded by a medical professional when a patient delivers a controller back to the prescribing medical professional, e.g., after the prescribed time allotment for the controller has depleted. The log can be stored in a variety of data formats or files, such as separated values, as a text file, or as a spreadsheet to enable a medical professional to display activity reports for the controller. In some implementations, the microcontroller is configured to log information related to errors associated with coil connections, electrical characteristics of the coil over time, dates and times of use of the therapy system, battery charge durations and discharge traditions, and inductance measurements or other indications of a coil being placed in contact with a patient's body. The microcontroller can provide log data or the log file to a medical professional using a USB port or other mode of communication to allow the medical professional to evaluate the quality and/or function of the therapy system and the quantity and/or use of the therapy system by the patient. Notably, the microcontroller can be configured to log any disruptions in signal delivery and can log any errors, status messages, or other information provided to the user through user interface of the controller (e.g., using the LCD screen).

The microcontroller can be configured to use volatile memory to protect the content of the prescription file. In some implementations, the prescription file is encrypted when the microcontroller transfers the prescription file from an external source into non-volatile memory. The microcontroller can further be configured to only store decrypted versions of the content of the prescription file in volatile memory. By limiting the storage of decrypted content to volatile memory, the microcontroller and thus the controller can ensure that decrypted content is lost when power is removed from the microcontroller circuitry.

The microcontroller can be configured to execute additional security measures to reduce the likelihood that an unauthorized user will obtain the contents of the prescription file. For example, the microcontroller can be configured to only decrypt the prescription file after verifying that an authorized or legitimate coil and cable assembly has been connected to the controller. As described above, the coil and cable assembly can include an integrated circuit that may store one or more encrypted or not encrypted identifiers for the coil and cable assembly. In some implementations, the microcontroller is configured to verify that an authorized or prescribed coil and cable assembly are connected to the controller. The microcontroller may verify the authenticity of a coil and cable assembly by comparing the identifier from the integrated circuit of the coil and cable assembly with one or more entries stored in a lookup table in either volatile memory or non-volatile memory. In other implementations, the microcontroller is configured to acquire a serial number of the integrated circuit and measure electrical characteristics of the coil and cable assembly and perform a cryptographic function, such as a hash function, on a combination of the serial number and the electrical characteristics. Doing so may deter or prevent an unauthorized user from copying the contents of the integrated circuit of the coil and cable assembly into a duplicate integrated circuit associated with an unauthorized copy of a coil and cable assembly.

The microcontroller can be configured to delete the prescription file from volatile memory and from non-volatile memory in response to fulfillment of one or more predetermined conditions. For example, the microcontroller can be configured to delete the prescription file from memory after the controller has delivered the prescribed drug-simulating signals for a specific period of time, e.g., 14 days. In other embodiments, the microcontroller is configured to delete the prescription file from memory after the controller detects that the device housing the coil and cable assembly has been hacked. The microcontroller can be configured to delete the prescriptive file after only one coupling with an unauthorized coil and cable assembly or can be configured to delete the prescription file after a predetermined number of couplings with an unauthorized coil and cable assembly. In some implementations, the microcontroller is configured to monitor an internal timer and delete the prescription file, for example, one month, two months, or longer after the prescription file has been installed on the controller.

The microcontroller can delete the prescription file from volatile memory and from non-volatile memory in response to input from one or more sensors (e.g., of a wearable). A sensor can provide a signal to the microcontroller in response to a physical disruption of the housing of the controller. For example, the sensor can be a light sensor that detects visible and non-visible wavelengths within the electromagnetic spectrum. For example, the sensor can be configured to detect infrared, visible light, and/or ultraviolet light. Because the detection of light within the housing can be an indication of intrusion into the housing, the microcontroller can be configured to delete and/or corrupt the prescription file upon receipt of a signal from the sensor. In some implementations, the sensor is a light sensor. In other implementations, the sensor can be a pressure sensor, a capacitive sensor, a moisture sensor, a temperature sensor, or the like.

In response to detection of unauthorized use of the controller, or to increase the user-friendliness of the therapy system, the microcontroller can use various indicators or interfaces to provide information to a user. An example can include a graphical icon on a display device and an audible sound. The microcontroller can present the graphical icon and/or actuate the audible sound in response to user error, unauthorized tampering, or to provide friendly reminders of deviation from scheduled use of the therapy system. Multiple visual indicators (e.g., LEDs) of various types or colors can also be used. Additionally, although the audio generator can be a vibrating motor, or a speaker that delivers audible commands to facilitate use of the system by sight impaired persons.

The microcontroller can manipulate an interface to interact with a user. The interface can receive various commands from the microcontroller. In response to inputs received from the microcontroller, a screen can be configured to display various messages to a user. In some implementations, the screen displays messages regarding battery status, duration of prescription use, information regarding the type of prescription being administered, error messages, identification of the coil and cable assembly, or the like. For example, the screen can provide a percentage or a time duration of remaining battery power. The screen can also provide a text-based message that notifies the user that the battery charge is low or that the battery is nearly discharged. The screen can also be reconfigured to provide a name of a prescription (e.g., corresponding name of the physical drug) and/or a body part for which the prescription is to be used. The screen can also provide notification of elapsed-time or remaining-time for administration of a prescription. If no additional prescription time is authorized, the screen can notify the user to contact the user's medical professional.

The screen can be configured to continuously or periodically provide indications regarding the status of the connection between a coil and the controller. In some implementations, the screen can be configured to display statuses or instructions such as, “coil connected”, “coil not connected”, “coil identified”, “unrecognized coil”, “reconnect coil”, or the like. In some implementations, the screen can provide a graphical representation of a coil and flash the coil when the coil is connected properly or improperly. Alternatively, or additionally, the controller can monitor an impedance from the coil to detect a change, a possible removal, or loss of the coil from the area to be treated, and provide a corresponding error message. The interface, in other implementations, is a touch-sensitive screen that delivers information to the user in addition to receiving instructions or commands from the user. In some implementations, the microcontroller can be configured to receive input from hardware buttons and switches to, for example, power on or power off the controller. The switch on the device permits an on-off nature of therapy so that patients may selectively switch on and off their therapy if needed.

Signal generation circuitry may be used to drive the coil and cable assembly with the drug-simulating signals. The circuitry can include an audio coder-decoder, and output amplifier, and a current monitor. The audio coder-decoder can be used to convert digital inputs received from volatile memory, non-volatile memory, or from microcontroller into analog output signals useful for driving the coil and cable assembly. The audio coder-decoder may be configured to output the analog output signals to the output amplifier. In some implementations, the output amplifier is programmable so that the intensity or amplitude of the signals transmitted to the coil may be varied according to the treatment prescribed for the person.

Because the controller can connect with coils having different sizes, shapes, and numbers of windings, the output amplifier can be configured to adjust an intensity level of signals delivered to the coil so that each coil delivers a drug-simulating signal that is uniform between different coils, for a particular prescription. The coil dimensions and electrical characteristics can determine the depth and breadth of concentration of the magnetic field, so programmatically adjusting the output intensity of the output amplifier to deliver uniform drug-simulating signals can advantageously enable a medical professional to select a coil that is appropriate for a particular patient's body or treatment area, without concern for inadvertently altering the prescription. As described above, the controller can determine the dimensions and electrical characteristics of a coil by reading such information from the integrated circuit. The signal generation circuitry can be configured to use the dimensional and electrical characteristic information acquired from the coil to programmatically adjust the level of intensity of signals output by the output amplifier.

The output amplifier can include a low-pass filter that significantly reduces or eliminates output signals having a frequency higher than, for example, 50 kHz. In other implementations, the low-pass filter can be configured to significantly reduce or eliminate output signals having a frequency higher than a threshold range (e.g., 0-25 kHz). The signal generation circuitry uses the current monitor to determine electrical characteristics of the coil and cable assembly and/or to verify that output signal levels remain within specified thresholds. The signal generation circuitry may also include a connector that mates with the connector of the coil and cable assembly. The connector can provide the electrical interface between the microcontroller and the coil and cable assembly.

In an implementation, the system includes an interface that allows a user or operator to select desired files, how long to play the file, whether files can be mixed or concatenated to provide a “playlist” for playback, etc. Thus, the interface allows for several different cognates to be selected individually or in groups along with the duration of play. In some embodiments, the interface provides applications that are fixed to a specific cognate or sets of cognates intended to play serially. The output power is fixed and cannot be adjusted by the end user so as to avoid unintentional physiological effects.

### Enhancing Drug-Simulating Signals

The disclosed embodiments include techniques for improving the efficacy of drug-simulating signals and/or discovering a most efficacious signal. That is, signals that simulate particular drugs could be in very noisy environments. Thus, it would be beneficial to detect signals despite the noise and identify which of those signals is the most efficacious at producing a physiological effect on a person.

In one implementation, a signal can be enhanced by 1, 2, or more decibels of power across a spectrum between 0 and 6 kHz, and then drop off to a noise floor. In this implementation, the non-linear structure of the signal of interest resides within that bandwidth. As such, it would be desirable to remove any competing or interfering signals below, for example, 6 kHz.

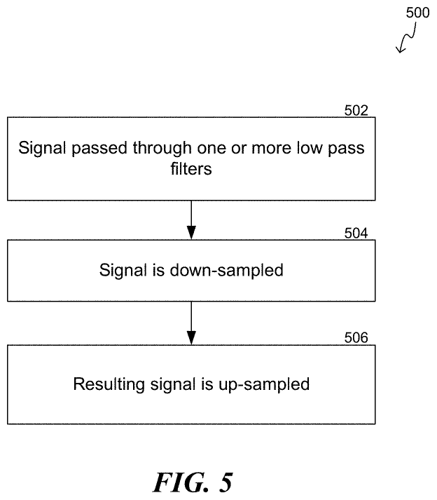

FIG. 5 illustrates a method 500 for enhancing a drug-simulating signal. The system can perform filtering and/or truncating to enhance only a specific portion of the original signal, where that portion provides the desired physiological effect.

At 502, the signal is passed through a 6 kHz and/or 7 kHz filter, which improves the efficacy of the signal considerably. At 504, the signal is down-sampled from about 44.1 kHz to 11 kHz or less, and then at 506 the result is up-sampled back up to 44.1 kHz. Doing so removes content within the signal above a noise threshold for a sampling frequency. This further provides a sharp roll-off at 79 decibels and the resulting signal has greater efficacy. Thus, the enhanced signal can provide increased onset and sensation of the produced physiological effect.

In one example, sampling rates include 11,025 samples per second (s/s), 16,384 s/s, 22,050 s/s, or 44,100 s/s. The signal can be “truncated,” meaning a signal is down sampled from a higher sampling rate to a lower one, for instance from 44,100 samples per second to 22,050 samples per second, and as low as 11,025 samples per second. Embodiments can also include low-pass filtering of various signals at 5 kHz and 6 kHz using analog or digital filters including Butterworth filters of 4 to 8 poles.

A file size for a signal is reduced by using this low-pass filter and down-sampling, assuming that the remaining file still provides the information needed. Here, the low-pass filter and down-sampling can remove unnecessary radio frequency (RF) energy from being transmitted into a biologic system. It is possible that unwanted radio energy located at higher frequencies can compete with frequencies responsible for the physiological effect of interest. In one example, the unnecessary RF energy may be referred to as “noise,” which can trigger an electromagnetic interference, and result in degrading the performance of the physiological effect of interest. Moreover, an amplified noise above target frequencies may reduce the signal-to-noise ratio of the frequencies of interest.

A secondary advantage of low sampling rates is significantly smaller file size. In one example, a static offset is employed to mitigate noise. Some embodiments use broadband Fourier analysis to increase power below approximately 6 kHz that are responsible for the increase in power and biologic activity. In some implementations, the system requires down sampling a signal and resampling the signal back to a 44,100 Hz rate. For example, some implementations cannot encode and/or decode signals that have a sampling rate lower than 44,100 Hz. Other embodiments include signals that can be encoded to accept multiple sample rates and bit rates.

In other implementations, and as noted above, the signal generation circuitry can also include an inductance detection circuitry. The inductance detection circuitry can be configured to detect changes in the coil inductance. The coil inductance changes when the coil is brought into proximity of a patient's body. By monitoring coil inductance, the signal generation circuitry and the controller can track and record (i.e., log) a patient's use of the therapy system. For example, if a medical professional prescribes 10 hours of use of the therapy system, but the controller only logs three hours of use of the therapy system 100, the medical professional may be in a better position to evaluate a patient's improving, non-improving or deteriorating condition. In some implementations, the inductance detection circuitry is implemented as a source follower circuit.

The controller can include a power control circuitry for receiving and regulating power to the controller. The power control circuitry includes a power input circuitry and power regulation circuitry. The power input circuitry can include a connector (e.g., a micro-USB connector) to receive power from an external source for recharging a battery. The power input circuitry can also include a charging circuit that monitors a voltage level of the battery and electrically decouples the battery from the connector when the battery is sufficiently charged. The power regulation circuitry can be used to convert a voltage level of the battery to a lower voltage for use by the various circuits of the controller. For example, when fully charged, the battery may have an approximate voltage between 4.2 and 5 volts, whereas the microcontroller may have an upper voltage threshold of 3.5 volts. The power regulation circuitry can be configured to convert the higher voltage of the battery (e.g., 4.2 volts) to a lower voltage, (e.g., 3.3 volts) that is usable by the electronic devices of the controller.

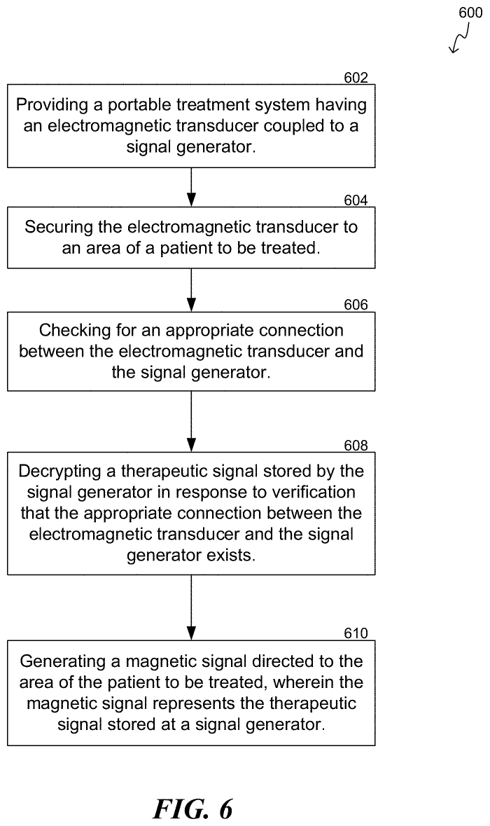

FIG. 6 illustrates a method 600 of operating a system configured to provide magnetic field therapy that is non-invasive, non-thermal, and mobile.

At 602 an electromagnetic transducer is activated by a controller. The electromagnetic transducer can include a coil having various shapes and sizes selected according to the size or condition of an ailment to be treated.

At 604 the electromagnetic transducer is secured to an area of the person to be treated. The transducer can be secured as the wearables is secured or by using elastic bandages, gauze, tape, or the like.

At 606, the controller checks for an appropriate connection to the electromagnetic transducer. The controller can be configured to verify an identification or electrical characteristics of the electromagnetic transducer, such as a resistance or impedance of the transducer to ensure that an appropriate transducer is coupled to the generator. In some implementations, the controller is configured to periodically monitor the electrical characteristics of the electromagnetic transducer to ensure that an appropriate connection is maintained. For example, if the signal generator detects an increase in resistance or decrease in inductance, the signal generator can be configured to cease delivery of signals to the electromagnetic transducer. The signal generator may cease delivery of signals when unexpected electrical characteristics are detected to protect the health and safety of the patient and to prevent unauthorized attempts to acquire generated signals. As discussed above, the signal generator can be configured to log the periodic checks of the electrical characteristics of the electromagnetic transducer and can provide the log data to a medical professional for review. Other security checks may be performed as described herein. In one implementation, a serial number or PIN is required as a security check for two or three factor authentication. A connected smart device could have a scheduling software that could prompt the user to press play or decline the use of a signal. The program associated with the delivery system could also require that a physician or health care worker provide an access code to the patient or user.

At 608 the controller decrypts a signal stored by the controller in response to verification that an appropriate connection between the electromagnetic transducer and the signal generator exists.

At 610 the electromagnetic transducer generates a magnetic signal directed to an area of the person. The magnetic signal is representative of the therapeutic signal stored at the signal generator. According to various implementations, the magnetic signal has a frequency in a range of 0 to 22 kHz.

In some implementations, a signal from a sample of a drug, biologic, or other molecule (chemical, biochemical, biologic), may be acquired by placing a sample in an electromagnetic shielding structure and by placing the sample proximate to at least one SQUID coupled to a magnetometer. The drug sample is placed in a container having both magnetic and electromagnetic shielding, where the drug sample acts as a signal source for molecular signals. Noise can be injected into the drug sample, in the absence of another signal, from another signal source at a noise amplitude sufficient to generate stochastic resonance, where the noise has a substantially uniform amplitude over multiple frequencies. Using the SQUID or the magnetometer, output radiation from the drug sample is detected and recorded as an electromagnetic time-domain signal composed of drug sample-source radiation superimposed on the injected noise in the absence of the other generated signal. The injecting of noise and detecting of the radiation may be repeated at each of multiple noise levels within a selected noise-level range until the drug sample source radiation is distinguishable over the injected noise. In some embodiments, the SQUID has a thermal baffle that is used to slow the boil-off of cryogenic Helium. In an application, it also provides an additional layer of electromagnetic shielding by adding an additional electromagnetic isolation of 3 to 6-dB.

### Helmholtz Configuration for Delivery of Drug-Simulating Signals

The drug-simulating signals can be delivered to a person via a system having a Helmholtz configuration with multiple signal generators (e.g., coils) that are configured to additively produce a signal that is targeted for an area between the coils. This array of signal generators is coordinated by a controller to generate and/or transmit signals that can collectively provide enough signal energy to cause a physiological effect on a person. The additive and targeted effect of using the Helmholtz configuration can involve placing the signal generators at different locations that are not physically in contact with the person. Moreover, the signal strength of any one signal generator can be kept relatively low despite being physically distant from the target person because the drug-simulating signal that the person receives could result from a combination of lower strength signals.

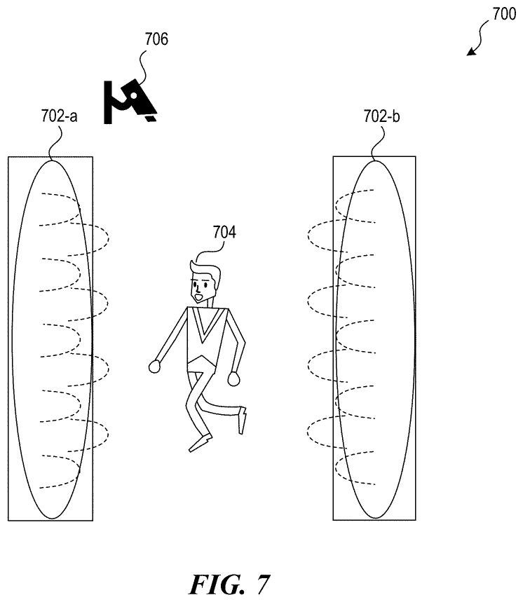

FIG. 7 illustrates a Helmholtz configuration of signal generators that can target drug-simulating signals. As shown, a Helmholtz configuration 700 has two identical electromagnetic coils 702-a and 702-b or solenoids oriented in parallel, positioned one radius apart with both coils operating in phase with one another. This design provides a high uniformity of field across the interior volume of space between the coils where a person 704 is positioned. Helmholtz configurations have the advantage of producing a uniformity of field over large areas as compared to single coils with fields that rapidly diminish at an inverse square of the distance.

A Helmholtz configuration can produce even larger volumes of uniformity by adding additional coil(s) in the center of a Helmholtz array. Adding multiple center coils can be applied to further increase the volume, or extend the physical length of uniformity (e.g., Maxwell coils). In one implementation, the Helmholtz array could be used to apply an ultra-low Radio Frequency Energy (u/RFE) field by installing coils on either side of a small room or portions of a room where people congregate. Smaller arrays could be positioned repetitively throughout a room or area where people move about, prolonging their exposure to a single cognate or experience as they move about. For example, Helmholtz arrays could be used with a u/RFE cognate to alter the mental state of customers in a department store by providing a sense of relaxation, happiness or other emotion that entices someone to stay and shop. Helmholtz arrays might also be used to reduce agitation in groups of people or produce emotions that might encourage people to leave an area.

In addition, a motion detector 706 (e.g., a camera) can be used to track the location of the person 704 such that the multiple signal generators can be dynamically adapted based on the person 704's position and thereby keep the signal strength of each individual signal generator relatively low instead of needing to flood the entire space with constant signals.

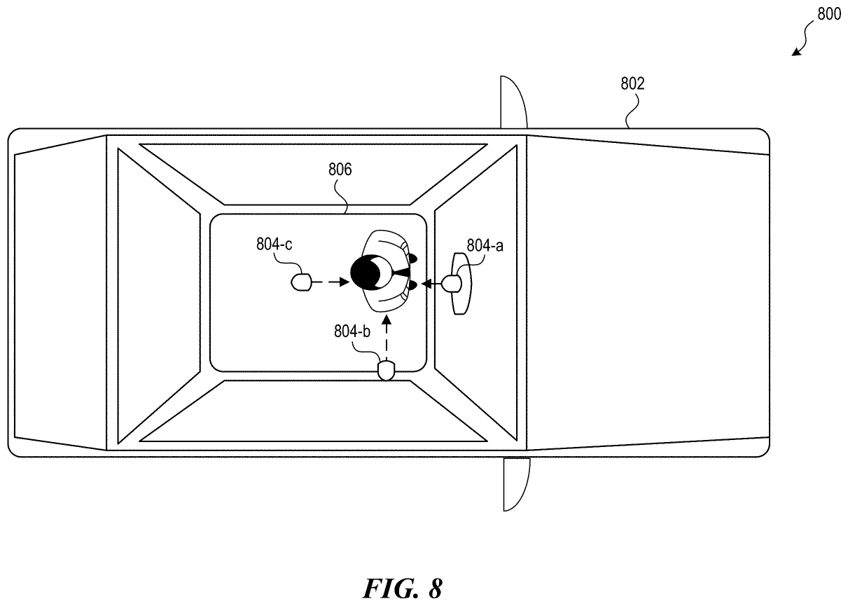

FIG. 8 is a diagram 800 that illustrates an array of devices including signal generators located in a vehicle 802. The signal generators 804-a, 804-b, and 804-c (collectively “signal generators 804”) are not physically worn by a driver 806 but still deliver drug-simulating signals to the driver 806. As shown, signal generators 804 are positioned on three locations of the vehicle 802 being driven by the driver 806. The locations include the front windshield, rear windshield, and passenger side window. The coils can be designed to rotate and move to provide accurate placement and steer the drug-stimulating signals. For example, as components of one or more arrays, the signal generators 804 can steer drug-stimulating signals to persons such as the driver 806 or a passenger. As such, embodiments can delivery drug-simulating signals that can keep the driver 806 alert by producing a physiological effect of caffeine. While shown in a vehicle, In other implementations, the array of devices can be positioned in other structures. For example, the coils of a Helmholtz configuration can be embedded in structures such as a chair. A chair fitted with Helmholtz coils can provide treatment of CNS disorders. These or similar structures may have utility beyond the treatment of CNS disorders. For example, chairs or recliners may be equipped with Helmholtz arrays for general wellness and entertainment purposes.

### Machine Learning Capabilities

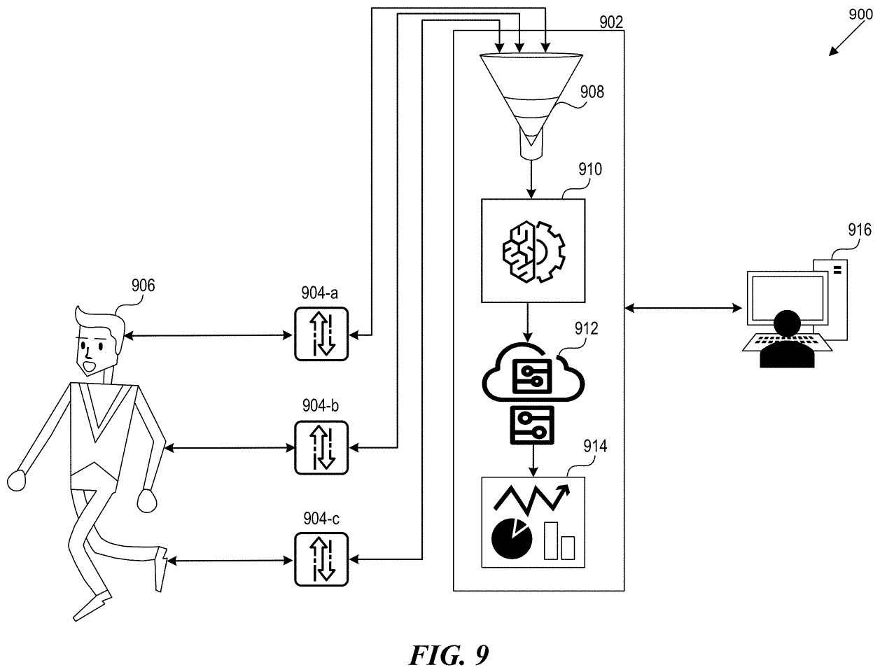

FIG. 9 is a block diagram that illustrates a system including machine learning (ML) capabilities that improve the efficacy of drug-simulating signals. As shown, the system 900 includes a simulator 902 that is configured to process recordings of electrostatic potentials of the chemical, biochemical, or biological molecules associated with physiological effects caused by drugs or other substances.

The system 900 can include a combination of sensors and/or signal generators 904-a, 904-b, and 904-c disposed on or near a person 906. The sensors and/or signal generators 904-a through 904-c generate drug-simulating signals and/or measure physical properties of the person 906 including, for example, temperatures and any other measures that are indicative of the physiological effect of a drug or a drug-simulating signal. For example, the sensor data can be used to determine values indicative of a particular physiological state of the person 906.

The drug-simulating signals and sensor data are communicated over communication channels between the person 906 and the simulator 902. The sensor data can be periodically generated by the sensors 904-a through 904-c and/or periodically communicated to the simulator 902, which is configured to, for example, perform data mining including standardization, thus enabling analytics based on a simulation of the physiological effects on the person 906 in response to the drug-simulating signals. As such, the simulator 902 includes a data mining component 908, which implements processes for extracting and discovering patterns in datasets. The data mining component 908 has an overall goal of extracting data from datasets for transforming the extracted data into information that has a comprehensible structure for further use. The data mining can also involve database and data management, data pre-processing, model and inference considerations, metrics processing, complexity considerations, post-processing of discovered structures, visualization, etc.

The data mining component 908 can standardize data to have a common format or use a common taxonomy. Further, the sensor data can be classified for a particular performance metric. For example, the data mining component 908 can extract temperature measurements and classify the sensor data in terms of the locations of the sensors 904-a through 904-c that captured the temperature measurements, characteristics or demographics of the person 906, and/or other dimensions that can be used for classifying the data. In another implementation, the data mining component 908 performs data aggregation to compile data extracted from datasets and combined to prepare new datasets to optimize processing by other components of the simulator 902.

The simulator 902 includes a modeling component 910 such as ML modeling algorithms, which constructs computer algorithms that automatically improve themselves through experience and with additional data. For example, the ML algorithms can build a model, or training dataset based on sample sensor data to enable forecasting or making predictions or decisions as to the efficacy of a drug-simulating signals based on a simulated change. In another implementation, the modeling component 910 includes a cataloging function that is created and updated manually based on the sensor data such that the impact of a drug-simulating signals on a target physiological system can be predicted when comparing data against the catalog of sensor data and performing interpolation or other numerical, computational, or statistical methods to extrapolate how a change can impact the performance of a drug-simulating signal.

The simulator 902 includes a simulation component 912 configured to cause the state of one or more physiological systems in response to a change in any of those systems or of drug-simulating signals. For example, the simulation component 912 can simulate the influence of the same drug-simulating signals on people of different weights or ages. The simulations require the use of models generated by the model component 910, which represent key characteristics or behaviors of a targeted physiological system or process, whereas the simulation represents the evolution of the model over time or in response to changes.

The simulator 902 includes an analytics component 914 that can generate and/or administer a network portal. An example includes an online web-based portal that can display the simulation or associated data in visualizations or other user-friendly features that enable end users 916 to investigate simulations and learn procedures for improving the efficacy of drug-simulating signals. The analytics component 914 enables continuous iterative exploration and investigation of past performance to predict future performance in different scenarios. The analytics component 914 utilizes statistical methods to form a data-driven understanding of a target physiological system and associated systems, and to develop new insights into the performance of the multiple physiological systems. The analytics component 914 makes extensive use of tools and analytical modeling and numerical analysis, including explanatory and predictive modeling, as well as fact-based management to drive decision-making.

The end user 916 includes any end user devices operable by users or computing devices that are authorized to access components or data processed by the simulator 902. In one example, the end user 916 is assigned a role that grants access to one, any, or all components of the simulator 902. For example, an end user with a reviewer role is only permitted access to the analytics component 914 whereas an end user with an administrator role is permitted to access all of the components of the simulator 902 to, for example edit the model of the modeling component 910 or modify how datasets are aggregated by the data mining component 908.

In a testing scenario, different drug-simulating signals are prototyped in an environment where each creature is exposed to a different type of drug-simulating signal, or different combinations of types, as well as different intensities, to determine the efficacy of signals to achieve a desired physiological effect. A camera could be positioned to monitor each of the creatures and provide feedback regarding their states under particular conditions. As such, the system 900 can optimize to determine the most efficacious drug-simulating signals. The system 900 can include a number of sensors 904-a through 904-c required for 3D environment measurements (e.g., 8 sensors) to simulate 3D modulations on surface potential of a molecule. As such, the system 900 can automate discovery of the most efficacious signals and provide improved prost-processing that automates batch processing of signals. For a test, for example, the system 900 can process multiple signals in batch processing to determine which signal between at or below 6 kHz band caused the most efficacious physiological effect.

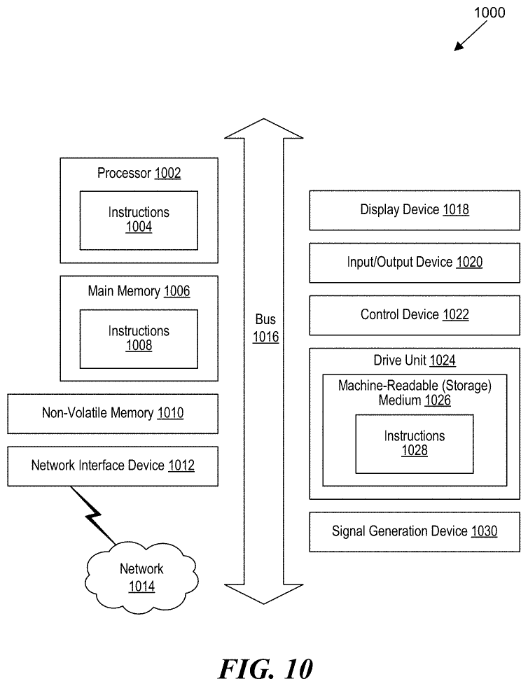

FIG. 10 is a block diagram that illustrates an example of a computer system 1000 in which at least some operations described herein can be implemented. As shown, the computer system 1000 can include: one or more processors 1002, main memory 1006, non-volatile memory 1010, a network interface device 1012, video display device 1018, an input/output device 1020, a control device 1022 (e.g., keyboard and pointing device), a drive unit 1024 that includes a storage medium 1026, and a signal generation device 1030 that are communicatively connected to a bus 1016. The bus 1016 represents one or more physical buses and/or point-to-point connections that are connected by appropriate bridges, adapters, or controllers. Further, the computer system 1000 may include common components, such as cache memory (not shown). Additionally, the computer system 1000 is intended to illustrate a hardware device where components illustrated or described relative to the examples of the figures and any other components described in this specification can be implemented.

The computer system 1000 can take any suitable physical form. For example, the computing system 1000 can share a similar architecture as that of a server computer, personal computer (PC), tablet computer, mobile telephone, game console, music player, wearable electronic device, network-connected (“smart”) device (e.g., a television or home assistant device), AR/VR systems (e.g., head-mounted display), or any electronic device capable of executing a set of instructions that specify action(s) to be taken by the computing system 1000. In some implementations, the computer system 1000 can be an embedded computer system, a system-on-chip (SOC), a single-board computer system (SBC) or a distributed system such as a mesh of computer systems, or include one or more cloud components in one or more networks. Where appropriate, one or more computer systems 1000 can perform operations in real-time, near real-time, or in batch mode.

The network interface device 1012 enables the computing system 1000 to mediate data in a network 1014 with an entity that is external to the computing system 1000 through any communication protocol supported by the computing system 1000 and the external entity. Examples of the network interface device 1012 include a network adaptor card, a wireless network interface card, a router, an access point, a wireless router, a switch, a multilayer switch, a protocol converter, a gateway, a bridge, bridge router, a hub, a digital media receiver, and/or a repeater, as well as all wireless elements noted herein.

The memory (e.g., main memory 1006, non-volatile memory 1010, machine-readable medium 1026) can be local, remote, or distributed. Although shown as a single medium, the machine-readable medium 1026 can include multiple media (e.g., a centralized/distributed database and/or associated caches and servers) that store one or more sets of instructions 1028. The machine-readable (storage) medium 1026 can include any medium that is capable of storing, encoding, or carrying a set of instructions for execution by the computing system 1000. The machine-readable medium 1026 can be non-transitory or comprise a non-transitory device. In this context, a non-transitory storage medium can include a device that is tangible, meaning that the device has a concrete physical form, although the device can change its physical state. For example, despite this change in state, the term non-transitory refers to a device remaining tangible.

Although implementations have been described in the context of fully functioning computing devices, the various examples are capable of being distributed as a program product in a variety of forms. Examples of machine-readable storage media, machine-readable media, or computer-readable media include: (1) recordable-type media such as volatile and non-volatile memory devices 1010; (2) removable flash memory; (3) hard disk drives; (4) optical disks; (5) and transmission-type media, such as digital and analog communication links.

In general, the routines executed to implement examples herein can be implemented as part of an operating system or a specific application, component, program, object, module, or sequence of instructions (collectively referred to as “computer programs”). The computer programs typically comprise one or more instructions (e.g., instructions 1004, 1008, 1028) set at various times in various memory and storage devices in computing device(s). When read and executed by the processor 1002, the instruction(s) cause the computing system 1000 to perform operations to execute elements involving the various aspects of the disclosure.

### Definitions

The terms below generally have the following definitions unless indicated otherwise. Such definitions, although brief, will help those skilled in the relevant art to more fully appreciate aspects of the invention based on the detailed description provided herein. Other definitions are provided above. Such definitions are further defined by the description of the invention as a whole (including the claims) and not simply by such definitions.

“Radio frequency energy” refers to magnetic fields having frequencies in the range of approximately 0 Hz to 22 kHz.

“Magnetic shielding” refers to shielding that decreases, inhibits or prevents passage of magnetic flux as a result of the magnetic permeability of the shielding material.

“Electromagnetic shielding” refers to standard Faraday electromagnetic shielding, or other methods to reduce passage of electromagnetic radiation.

“Faraday cage” refers to an electromagnetic shielding configuration that provides an electrical path to ground unwanted electromagnetic radiation, thereby quieting an electromagnetic environment.

“Time-domain signal” or “time-series signal” refers to a signal with transient signal properties that change over time.

“Sample-source radiation” refers to magnetic flux or electromagnetic flux emissions resulting from the molecular motion of a sample, such as the rotation of a molecular dipole in a magnetic field. Because sample source radiation may be produced in the presence of an injected magnetic-field stimulus, it may also be referred to as “sample source radiation superimposed on injected magnetic field stimulus.”

“Stimulus magnetic field” or “magnetic-field stimulus” refers to a magnetic field produced by injecting (applying) to magnetic coils surrounding a sample, one of a number of electromagnetic signals that may include (i) white noise, injected at voltage level calculated to produce a selected magnetic field at the sample of between 0 and 1 G (Gauss), (ii) a DC offset, injected at a voltage level calculated to produce a selected magnetic field at the sample of between 0 and 1 G, and/or (iii) sweeps over a low-frequency range, injected successively over a sweep range between at least between 0 and 1 kHz, and at an injected voltage calculated to produce a selected magnetic field at the sample of between 0 and 1 G. The magnetic field produced at the sample may be readily calculated using known electromagnetic relationships, knowing a shape and number of windings in an injection coil, a voltage applied to coils, and a distance between the injection coils and the sample.

A “selected stimulus magnetic-field condition” refers to a selected voltage applied to a white noise or DC offset signal, or a selected sweep range, sweep frequency and voltage of an applied sweep stimulus magnetic field.

“White noise” refers to random noise or a signal having simultaneous multiple frequencies (e.g., white random noise or deterministic noise). Several variations of white noise and other noise may be utilized. For example, “Gaussian white noise” is white noise having a Gaussian power distribution.

“Stationary Gaussian white noise” is random Gaussian white noise that has no predictable future components.

“Structured noise” is white noise that may contain a logarithmic characteristic which shifts energy from one region of the spectrum to another, or it may be designed to provide a random time element while the amplitude remains constant. These two represent pink and uniform noise, as compared to truly random noise which has no predictable future component.

“Uniform noise” refers to white noise having a rectangular distribution rather than a Gaussian distribution.

“Frequency-domain spectrum” refers to a Fourier frequency plot of a time-domain signal.

“Spectral components” refers to singular or repeating qualities within a time-domain signal that can be measured in the frequency, amplitude, and/or phase domains. Spectral components will typically refer to signals present in the frequency domain.

### Examples

Several aspects of the present technology are set forth in the following examples:

1. A method of producing a drug-simulating signal to simulate a physiological effect of a drug on a living organism, the method comprising:
measuring an electrostatic potential associated with a physiological system of the living organism under effect of the drug;
recording the measurement of the electrostatic potential of the physiological system to a memory of a closed system;
generating the drug-simulating signal that is configured based on the recording of the measurement of the electrostatic potential of the physiological system,
   wherein the drug-simulating signal is controlled with an amplifier circuit of the closed system, and
   wherein the drug-simulating signal includes an electromagnetic signal configured to simulate the effect of the drug on the living organism;
causing the amplifier circuit of the closed system to manipulate the drug-simulating signal based on feedback including measures of electrostatic potentials associated with the physiological system while being radiated with the drug-simulating signal to cause a desired physiological effect;
controlling delivery of the drug-simulating signal in the closed system in response to the feedback and based on a computer program stored in the memory within the closed system; and
responding to the feedback collected by the closed system to dynamically adapt efficacy of the drug-simulating signal toward the desired physiological effect.

2. The method of example 1, wherein the closed system comprises a wearable device, a handheld device, or a combination thereof.

3. The method of example 1, wherein the drug-simulating signal is enhanced by filtering and/or truncating a portion of the drug-simulating signal, wherein the portion causes the physiological effect.

4. The method of any one of examples 1-3, wherein the drug-simulating signal causing the physiological effect is filtered through a 6 kHz filter and/or a 7 kHz filter.

5. The method of any one of example 1-3, wherein the drug-simulating signal yielding the physiological effect is (1) down-sampled from about 44.1 kHz to 11 kHz or less, and (2) up-sampled to 44.1 kHz.

6. The method of any one of example 1 or examples 3-5, wherein a low-pass filter and down-sampling removes an unnecessary radio frequency (RF) energy from being radiated on the living organism during delivery of the drug-simulating signal to cause the physiological effect.

7. The method of example 6, wherein the unnecessary RF energy competes with a frequency responsible for the physiological effect.

8. The method of example 6, wherein the low-pass filter and down-sampling reduces a file size for the drug-simulating signal.

9. A method of optimizing a drug-simulating signal based on data obtained following a delivery of the drug-simulating signal to a living organism, the method comprising:
detecting efficacy of a delivered drug-simulating signal to the living organism by:
   generating one or more drug-simulating signals to deliver to the living organism;
   measuring a physiological effect of the delivered drug-simulating signal on the living organism by a sensor; and
improving the efficacy of the delivered drug-simulating signal to the living organism by a machine learning model comprising:
   creating a training dataset from the physiological effect data collected by the sensor;
   enabling the drug-simulating signal to make predictions or decisions based on the training dataset; and
   adapting dynamically to improve the efficacy of the drug-simulating signal.

10. The method of example 9, wherein improving the efficacy of the delivered drug-simulating signal for producing the physiological effect in the living organism, is performed by a system comprising a simulator configured to process a recording of an electrostatic potential for a drug and/or other substance collected by the sensor.

11. The method of example 10, wherein the drug-simulating signal corresponds to an electromagnetic signal that causes the physiological effect.

12. The method of example 11, wherein the electrostatic potential is generated from a molecule selected from the group consisting of:
a chemical molecule,
a biochemical molecule, and
a biological molecule.

13. The method of example 9, wherein generating one or more drug-simulating signals to deliver to the living organism is performed by a signal generator.

14. The method of any one of examples 9 or 10, wherein the sensor measures a physical property of the living organism from the delivered drug-simulating signal producing in the physiological effect.

15. The method of example 14, wherein the physical property of the living organism comprises a property indicative of the physiological effect of the drug-simulating signal.

16. The method of example 15, wherein the property indicative of the physiological effect of the drug-simulating signal is a temperature of the living organism.

17. The method of example 10, wherein a communication channel communicates the drug-simulating signal between the living organism and the simulator.

18. The method of any one of examples 9, 10, or 14, wherein the sensor comprises one or more sensors selected from the group consisting of:
a magnetometer,
a proximity sensor,
a barometer,
a gyroscope, and
an accelerometer.

19. The method of example 9, wherein creating the training dataset from the physiological effect data collected by the sensor to improve the efficacy of the drug-simulating signal is performed by a modeling component.

20. The method of example 19, wherein the modeling component comprises forecasting decisions on the efficacy of the drug-simulating signal.

21. The method of example 20, wherein the forecasting decisions is based on a simulated change by a simulation component.

22. The method of example 10, wherein the simulator further comprises an analytics component, which administers a network portal.

23. The method of example 22, wherein the analytics component comprises a continuous iterative exploration and investigation of a past performance to forecast a future performance during a different event.

24. A device configured to deliver a drug-simulating signal, wherein delivery of the drug-simulating signal is radiated to a living organism to cause a physiological effect in the living organism, the device comprising:

a plurality of integrated circuit chips,
a plurality of sensors,
a communications circuitry configured to support a wireless communications protocol, or
any combination thereof.

25. The device of example 24, wherein the plurality of sensors comprises one or more sensors selected from the group consisting of:
a magnetometer,
a proximity sensor,
a barometer,
   a gyroscope, and
an accelerometer.

26. The device of example 24, wherein the device is configured to select an appropriate drug-simulating signal through a support wireless communications protocol to implement the physiological effect in the living organism.

27. The device of example 26, wherein the communications protocol comprises Bluetooth, Wi-Fi, satellite navigation, or any combination thereof.

28. The device of example 24, wherein the device is a wearable device.

29. The device of example 28, wherein the device comprises one or more devices selected from the group consisting of:
a smartwatch,
a smart eyewear, and
a wearable display device.

30. A controller to distribute and regulate a drug-simulating signal to a signal generator comprises:
a housing,
a processor,
a memory,
a visual and audio interface, or
any combination thereof.

31. The controller of example 30, wherein the controller further comprises a microcontroller circuitry and a signal generation circuitry.

32. The controller of example 31, wherein the microcontroller circuitry is configured to operate the controller.

33. The controller of example 31, wherein the microcontroller circuitry comprises:
a microprocessor,
a reset circuit, and
a volatile memory.

34. The controller of example 33, wherein the microprocessor comprises a plurality of analog and/or digital communication pins for communication inside and outside of the housing.

35. The controller of example 34, wherein the microprocessor further comprises:
a USB connector,
a display connector to a visual interface, and
an audio connector to an audio interface.

36. The controller of example 31, wherein the signal generation circuitry is configured to drive a coil and cable assembly with the drug-simulating signal.

37. The controller of example 36, wherein the signal generation circuitry comprises:
an audio coder-decoder,
an output amplifier, and
a current monitor.

38. The controller of example 37, wherein the audio coder-decoder is configured to output an analog output drug-simulating signal to the output amplifier.

39. The controller of example 37, wherein the output amplifier comprises a low-pass filter.

40. The controller of example 37, wherein the output amplifier is programmable.

41. The controller of example 37, wherein the output amplifier is further configured to adjust an intensity level of an input drug-simulating signal received by a coil of the coil and cable assembly.

42. The controller of example 37, wherein the current monitor determines an electrical characteristic of the coil and cable assembly.

43. The controller of example 37, wherein the current monitor verifies that a drug-simulating signal level remains within a specified frequency threshold.

44. A method for manufacturing a coil and cable assembly for use during non-intrusive delivery of a drug-simulating signal to simulate a physiological effect in a living organism, the method comprising:
encapsulating a coil in a flexible composite material;
coupling a connector to the coil;
coupling an integrated circuit to the coil, the connector, and a cable; and
embedding a memory storage in the integrated circuit.

45. The method of example 44, wherein coupling the integrated circuit to the cable further comprises a plurality of conductors delivering signals between the connector and the coil, and providing mechanical strain relief to the plurality of conductors.

46. The method of example 44, wherein the memory storage is configured to identify an electrical characteristic of the integrated circuit, the connector, the cable, and/or the coil.

47. The method of example 44, wherein the coil and cable assembly is configured to be encapsulated in a wearable device.

### CONCLUSION

The system described herein transduces a specific molecule signal to effect a specific charge pathway and may be configured to deliver the effect of chemical, biochemical or biologic therapy to a patient and treat an adverse health condition, without the use of drugs, alternative therapies, etc. For example, the system can transduce RNA sequence signals to regulate metabolic pathways and protein production, both up regulation and down regulation.

The system provides numerous other benefits. The system is scalable to provide treatment to a variety of patient regions. The coil, cable and connector are disposable, or the device as a whole with the controller, are preferably provided for a limited therapeutic session and for one prescription, so that the device and coil are not to be reused, thereby preventing cross contamination, etc. The switch on the device permits an on-off nature of therapy so that patients may selectively switch on and off their therapy if needed.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or” in reference to a list of two or more items, that word covers all of the following interpretations of the word: any of the items in the list, all of the items in the list, and any combination of the items in the list.

The above detailed description of embodiments of the invention is not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. For example, while processes or blocks are presented in a given order, alternative embodiments may perform routines having steps, or employ systems having blocks, in a different order, and some processes or blocks may be deleted, moved, added, subdivided, combined, and/or modified. Each of these processes or blocks may be implemented in a variety of different ways. Also, while processes or blocks are at times shown as being performed in series, these processes or blocks may instead be performed in parallel, or may be performed at different times.

The teachings of the invention provided herein can be applied to other systems, not necessarily the system described above. The elements and acts of the various embodiments described above can be combined to provide further embodiments.

All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

These and other changes can be made to the invention in light of the above Detailed Description. While the above description details certain embodiments of the invention and describes the best mode contemplated, no matter how detailed the above appears in text, the invention can be practiced in many ways. Details of the signal processing system may vary considerably in its implementation details, while still being encompassed by the invention disclosed herein.

As noted above, the particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless the above Detailed Description section explicitly defines such terms. Accordingly, the actual scope of the invention encompasses not only the disclosed embodiments, but also all equivalent ways of practicing or implementing the invention under the claims.

1. A method of producing a drug-simulating signal to simulate a physiological effect of a drug on a living organism, the method comprising:
measuring an electrostatic potential associated with a physiological system of the living organism under effect of the drug;
recording the measurement of the electrostatic potential of the physiological system to a memory of a closed system;
generating the drug-simulating signal that is configured based on the recording of the measurement of the electrostatic potential of the physiological system,
wherein the drug-simulating signal is controlled with an amplifier circuit of the closed system, and
wherein the drug-simulating signal includes an electromagnetic signal configured to simulate the effect of the drug on the living organism;
causing the amplifier circuit of the closed system to manipulate the drug-simulating signal based on feedback including measures of electrostatic potentials associated with the physiological system while being radiated with the drug-simulating signal to cause a desired physiological effect;
controlling delivery of the drug-simulating signal in the closed system in response to the feedback and based on a computer program stored in the memory within the closed system; and
responding to the feedback collected by the closed system to dynamically adapt efficacy of the drug-simulating signal toward the desired physiological effect.

2. The method of claim 1, wherein the closed system comprises a wearable device, a handheld device, or a combination thereof.

3. The method of claim 1, wherein the drug-simulating signal is enhanced by filtering and/or truncating a portion of the drug-simulating signal, wherein the drug-simulating signal causing the physiological effect is filtered through a 6 kHz filter and/or a 7 kHz filter, and wherein the portion causes the physiological effect.

4. (canceled)

5. The method of claim 3, wherein the drug-simulating signal yielding the physiological effect is (1) down-sampled from about 44.1 kHz to 11 kHz or less, and (2) up-sampled to 44.1 kHz.

6. The method of claim 5, wherein a low-pass filter and down-sampling removes an unnecessary radio frequency (RF) energy which competes with a frequency responsible for the physiological effect, from being radiated on the living organism during delivery of the drug-simulating signal to cause the physiological effect.

7. (canceled)

8. The method of claim 6, wherein the low-pass filter and down-sampling reduces a file size for the drug-simulating signal.

9. A method of optimizing a drug-simulating signal based on data obtained following a delivery of the drug-simulating signal to a living organism, the method comprising:
detecting efficacy of a delivered drug-simulating signal to the living organism by:
generating one or more drug-simulating signals to deliver to the living organism;
measuring a physiological effect of the delivered drug-simulating signal on the living organism by a sensor; and
improving the efficacy of the delivered drug-simulating signal to the living organism by a machine learning model comprising:
creating a training dataset from the physiological effect data collected by the sensor;
enabling the drug-simulating signal to make predictions or decisions based on the training dataset; and
adapting dynamically to improve the efficacy of the drug-simulating signal.

10. The method of claim 9, wherein improving the efficacy of the delivered drug-simulating signal for producing the physiological effect in the living organism, is performed by a system comprising:
corresponding the drug-simulating signal to an electromagnetic signal that causes the physiological effect;
configuring a simulator to process a recording of an electrostatic potential for a drug and/or other substance collected by the sensor;
communicating the drug-simulating signal between the living organism and the simulator through a communication channel; and
administering a network portal from the simulator comprising an analytics component.

11. (canceled)

12. The method of claim 10, wherein the electrostatic potential is generated from a molecule selected from the group consisting of:
a chemical molecule,
a biochemical molecule, and
a biological molecule.

13. The method of claim 9, wherein generating one or more drug-simulating signals to deliver to the living organism is performed by a signal generator.

14. The method of claim 9, wherein the sensor measures a physical property of the living organism indicative of the physiological effect of the delivered drug-simulating signal producing in the physiological effect.

15. (canceled)

16. The method of claim 14, wherein the property indicative of the physiological effect of the drug-simulating signal is a temperature of the living organism.

17. (canceled)

18. The method of claim 14, wherein the sensor comprises one or more sensors selected from the group consisting of:
a magnetometer,
a proximity sensor,
a barometer,
a gyroscope, and
an accelerometer.

19. The method of claim 9, wherein creating the training dataset from the physiological effect data collected by the sensor to improve the efficacy of the drug-simulating signal is performed by a modeling component.

20. The method of claim 19, wherein the modeling component comprises forecasting decisions on the efficacy of the drug-simulating signal.

21. The method of claim 20, wherein the forecasting decisions is based on a simulated change by a simulation component.

22. (canceled)

23. The method of claim 10, wherein the analytics component comprises a continuous iterative exploration and investigation of a past performance to forecast a future performance during a different event.

24.-29. (canceled)

30. A controller to distribute and regulate a drug-simulating signal to a signal generator comprises:
a housing,
a processor,
a memory,
a visual and audio interface, or
any combination thereof.

31. The controller of claim 30, wherein the controller further comprises:
a microcontroller circuitry configured to operate the controller, wherein the microcontroller circuitry comprises:
a microprocessor,
a reset circuit, and
a volatile memory; and
a signal generation circuitry configured to drive a coil and cable assembly with the drug-simulating signal, wherein the signal generation circuitry further comprises:
an audio coder-decoder, configured to output an analog output drug-simulating signal;
a programmable output amplifier, configured to receive the analog output drug-simulating signal to the output amplifier; and
a current monitor, configured to determine an electrical characteristic of the coil and cable assembly, and verify that a drug-simulating signal level remains within a specified frequency threshold.

32.-38. (canceled)

39. The controller of claim 31, wherein the programmable output amplifier comprises a low-pass filter, and is further configured to adjust an intensity level of an input drug-simulating signal received by a coil of the coil and cable assembly.

40.-47. (canceled)
